(19) United States
(12) Patent Application Publication
(10) Pub. No.: US 2026/0021271 A1
(43) Pub. Date: Jan. 22, 2026

(54) Care Set

(71) Applicant: VAN MEY MED, Molenbeek-Saint-Jean (BE)

(72) Inventor: Yassin YEMNI, Molenbeek-Saint-Jean (BE)

(73) Assignee: VAN MEY MED, Molenbeek-Saint-Jean (BE)

(21) Appl. No.: 19/269,248

(22) Filed: Jul. 15, 2025

(30) Foreign Application Priority Data
Jul. 16, 2024 (BE) 2024/5462

(51) Int. Cl. A61M 25/00; A61M 35/00

(52) U.S. Cl. CPC A61M 25/002; A61M 25/0084; A61M 35/003; A61M 2025/0089; A61M 2207/00

(57) ABSTRACT

A needle-free care set with a peripheral venous catheter as well as a preparation method of the care set prior to wound care are disclosed.

### CROSS-REFERENCE TO RELATED APPLICATION

This application claims priority to the patent application No. BE 2024/5462 filed Jul. 16, 2024, the disclosure of which is hereby incorporated by reference in its entirety.

### TECHNICAL FIELD

The disclosure concerns a care set with a peripheral venous catheter, in particular for wound care, and a method for preparing such a care set prior to wound care.

### BACKGROUND

A catheter is a well-known medical device consisting of a thin tube designed to be inserted into the lumen of a body cavity or blood vessel. It is used, for example, for drainage or infusion of fluids.

So called “peripheral venous” catheters are frequently used in various types of medical care. They are usually inserted percutaneously into a superficial vein, for example in a limb. Due to this preferred use, a peripheral venous catheter is typically supplied and packaged with a needle that passes through the tube. The practitioner inserts the catheter by inserting the needle coupled to the catheter and then withdrawing the needle.

In this document, the term “peripheral venous catheter” refers not only to the tube intended to be inserted into the superficial vein, but also to any elements specific to the catheter that may be attached to it, for example a reflux chamber from which the tube extends and, more optionally, attachment wings extending on either side of the reflux chamber.

A peripheral venous catheter can also be used with a syringe and physiological serum to clean a wound, for example prior to meshing. In this case, the practitioner uses a care set, for example a meshing set, consisting of a sterile sealed package enclosing various care tools such as a syringe, scissors, forceps and/or a stylet.

However, these cares require a great deal of handling, as the practitioner has to manipulate the catheter coupled to a needle and the care set. In this context, every such manipulation increases the risk of both medical error and undesirable contamination for the patient.

### SUMMARY

One object, among other, of this disclosure is to provide means of facilitating wound care and limiting the associated medical risks with respect to the prior art.

To this end, the present disclosure provides a care set having a peripheral venous catheter and being needle-free. The care set facilitates wound care and reduces the associated medical risks, as it no longer requires the catheter and needle to be handled at the same time, in particular to uncouple them from each other. The practitioner can therefore grasp the catheter directly without any further manipulation and use it with a syringe and physiological serum. The absence of a needle also reduces waste of the raw material from which it is made and significantly limits the cost of medical intervention by the practitioner.

The care set according to the disclosure preferably consists of a sterile sealed package enclosing one or more care tools, including the aforementioned catheter, and more preferably a syringe. Thus, the care tools essential for wound care are provided in a single package, which further reduces handling for the practitioner.

In one embodiment, the catheter is fitted in and/or attached to a dispensing end of the syringe. This limits the number of manipulations required of the practitioner. An fitting connection is easy to implement on a production line without the need for additional raw materials. The fitting connection and/or attachment is direct.

The fitting connection or attachment should preferably be made at the reflux chamber of the catheter, so that the dispensing end enters the reflux chamber. As the reflux chamber is generally conical in shape, the dispensing end fits into it, providing an essentially leak-proof connection between the functional interior of the syringe and the interior of the catheter tube.

Attachment, for example by means of a suitable attaching and/or sealing material such as an adhesive, has the advantage of securing the syringe more firmly and reliably to the catheter. It is also suitable in cases where a fitting connection is not possible due to the geometry of the reflux chamber and the dispensing end of the syringe.

Preferably, the catheter and syringe are cast in one piece. In particular, they are made from a material, typically a plastic. In this way, the resulting care tool can be used directly for wound care without any prior handling. Advantageously, it is solid because it is in one piece, with no gaps between the catheter and the syringe, so it is totally waterproof. This care tool can easily be produced by plastic moulding or any other known and suitable technique.

In one embodiment of the disclosure, the care tools include at least one of scissors, forceps, compresses, a sterile field, a wick and a stylet. All the care tools needed for wound care are available in a single care set, with a minimum of handling for the practitioner.

In one embodiment, the sterile sealed package enclosing the care tools is compartmentalized and comprises a first compartment which is oversized in relation to the dimensions of the catheter, preferably relative to all dimensions of the catheter, with the catheter being arranged in this compartment. This makes it easy for the practitioner to grasp the catheter regardless of the orientation of his or her hand in relation to the package and/or the compartment, which has the advantage of reducing the attempts to grasp and manipulation by the practitioner, as well as the time required for this purpose. Preferably, at least one of the care tools is arranged in the first compartment with the catheter.

This applies, for example, to syringes, particularly where the syringe and catheter are fixed and/or fitted together, but also in all other cases, so that these two essential wound care tools are available in a single compartment of a single package.

The care set proposed herein may alternatively be described in the context of the disclosure as consisting of a sterile closed package enclosing one or more care tools, including a peripheral venous catheter, the other possible care tool or tools being chosen from a syringe, scissors, forceps, compresses, a sterile field, a wick and a stylet. The relevant embodiments and advantages described above mutatis mutandis.

It should be noted that for purposes of this disclosure, the use of “including,” “comprising,” or “having” and variations thereof herein is meant to encompass the items listed thereafter and equivalents thereof as well as additional items. Similarly, the use in this document of the indefinite article “a”, “an”, or the definite article “the” to introduce an element does not exclude the presence of a plurality of these elements. The present specification may reference quantities and numbers. Unless specifically stated, such quantities and numbers are not to be considered restrictive. Generally, throughout the specification, terms of art may be used. These terms are to take on their ordinary meaning in the art from which they come, unless specifically defined herein or the context of their use would clearly suggest otherwise.

The present disclosure also provides a method for preparing a care set according to the disclosure prior to treating a wound. The care set of the method consists of a sterile sealed package enclosing one or more care tools, including:
a peripheral venous catheter comprising a tube extending from a reflux chamber, and
a syringe comprising a dispensing end.

The method comprises the following steps:
(i) opening the package;
(ii) taking the catheter and syringe;
(iii) fitting the dispensing end into the reflux chamber if this has not already been done or if the catheter is not attached to the dispensing end of the syringe;
(iv) withdrawing a quantity of care fluid into the syringe from a free end of the catheter tube.

This method is fully within the scope of the disclosure in that it facilitates wound care while limiting the associated medical risks. The manipulations offered by this care set are minimised and simplified as described above. This effect is accentuated when the syringe and catheter are fixed and/or cast in one piece, step (ii) being carried out by grasping.

The order of steps (iii) and (iv) and the optional nature of step (iii) is highly advantageous in this respect. In fact, since the care fluid can be drawn directly through said free end of the care tool tube formed by fitting the catheter onto the syringe, it is not necessary to draw the fluid through the dispensing end of the syringe, as is known in the prior art, before fitting the catheter. This reduces handling for the practitioner and makes it practical to supply the catheter already attached to the syringe in the sterile sealed package.

In the context of this method, the term “care fluid” is to be interpreted as any fluid used to care for the wound. It may be a care fluid or a wound cleansing fluid. The care fluid consists of physiological serum, i.e. a mixture of H₂O and NaCl defining an isotonic solution with 0.9% NaCl, according to a preferred embodiment. The features and benefits of the care set apply mutatis mutandis to the present method.

The disclosed subject matter is further introduced in the claims. As it will be understood by a skilled person from the present disclosure, any one of the embodiments presented in these claims can be considered alone or in combination. In particular, the dependency of the claims can be considered in a broader manner so that any one of the possible combinations of the claims—as far as they are technically possible and understood by the person skilled in the art, in particular in view of the present disclosure—are part of the present application.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1 shows a top view of a care set according to an embodiment of the disclosure;

FIG. 2 illustrates atop view of a catheter and a syringe fitted together as care tools of the care set according to one embodiment of the disclosure.

The drawings in the figures are not to scale. Similar elements are generally denoted by similar references in the figures. In the scope of this document, the same or similar elements may have the same references. In addition, the presence of reference numbers or letters in the drawings is not to be regarded as limiting, even when these numbers or letters are indicated in the claims.

### DETAILED DESCRIPTION

This section describes preferred embodiments of the present disclosure. The latter is described with particular embodiments and references to figures but the disclosure is not limited by them.

In the following description, details are set forth to provide a thorough understanding of representative embodiments of the present disclosure. It will be apparent to one skilled in the art, however, that the embodiments disclosed herein may be practiced without embodying all of the specific details. In some instances, well-known process steps or structural elements have not been described in detail in order not to unnecessarily obscure various aspects of the present disclosure. Further, it will be appreciated that embodiments of the disclosure may employ any combination of features described herein.

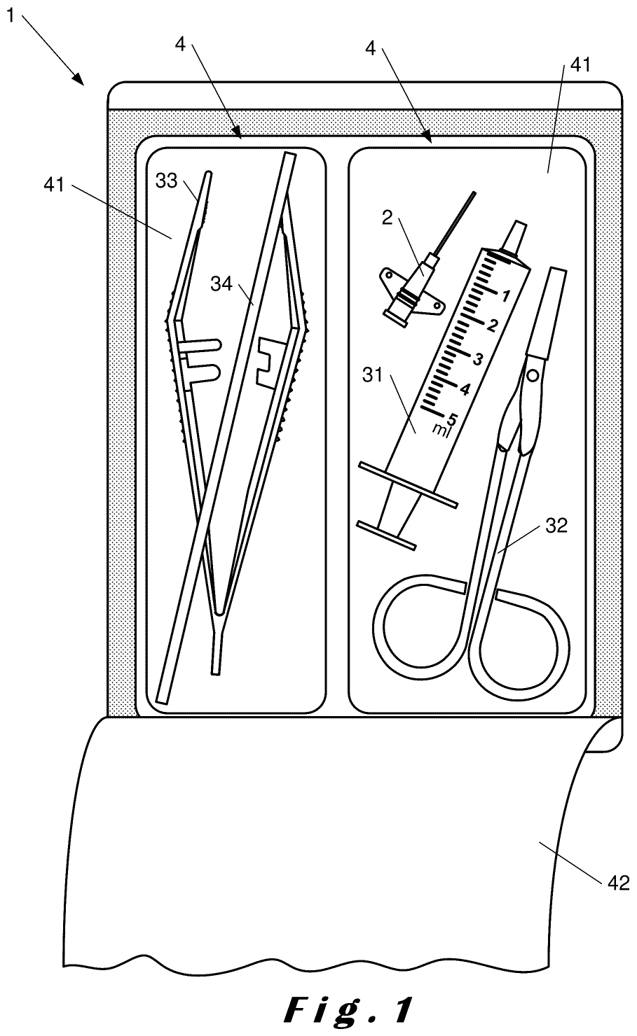

FIG. 1 illustrates a care set 1 according to the present disclosure in the form of a package 4 enclosing various care tools. The package 4 is originally closed by a top sheet 42 which is glued and/or removably adhered to the outer edges of a tray in which the care tools are arranged. The tray is divided into several compartments 41, each containing different care tools. All sterilised.

Care tools shown include peripheral venous catheter 2 which, in a known manner, comprises a thin tube extending from a reflux chamber to which attachment wings are attached. The care tools illustrated also include a syringe 31, scissors 32, forceps 33 and a stylet 34, although these are not limitative of the disclosure. The syringe 31 is enclosed in the same compartment 41 as the catheter 2, this compartment being oversized in all spatial dimensions, i.e. width, length and depth, compared to the dimensions of the catheter 2.

The care set 1 has no needles, and in particular no needles intended to cooperate with the catheter 2. It is suitable for wound care.

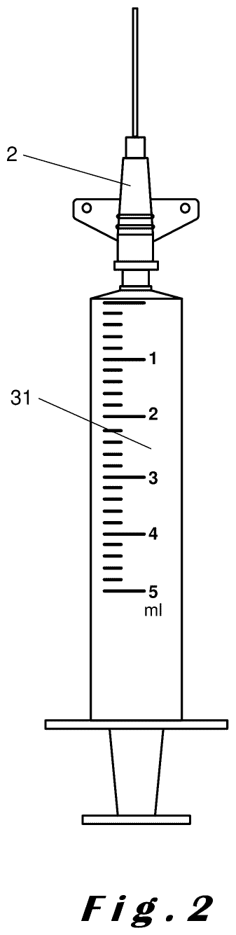

FIG. 2 shows the syringe 31 after its dispensing end has been fitted into the reflux chamber of the catheter 2, providing a fluid coupling between the inside of the catheter 2 tube and the inside of the syringe 31.

This configuration can be obtained from the care set 1 illustrated in FIG. 1 via step (iii) of the preparation method according to the disclosure. Alternatively, the catheter 2 can be supplied fitted in and/or attached to the dispensing end in the care set 1 as discussed in the disclosure of the disclosure. The illustration in FIG. 2 can also be supplied as a single plastic casting part.

The present application may reference quantities and numbers. Unless specifically stated, such quantities and numbers are not to be considered restrictive, but exemplary of the possible quantities or numbers associated with the present application. Also in this regard, the present application may use the term “plurality” to reference a quantity or number. In this regard, the term “plurality” is meant to be any number that is more than one, for example, two, three, four, five, etc. Unless otherwise indicated, the numerical values given as examples are given with a margin of error of 10%. For the purposes of the present disclosure, the phrase “at least one of A and B” is equivalent to “A and/or B” or vice versa, namely “A” alone, “B” alone or “A and B.”. Similarly, the phrase “at least one of A, B, and C,” for example, means (A), (B), (C), (A and B), (A and C), (B and C), or (A, B, and C), including all further possible permutations when greater than three elements are listed.

In the detailed description herein, references to “one embodiment”, “an embodiment”, “an example embodiment”, “one or more embodiments”, “some embodiments”, etc., indicate that the embodiment or embodiments described may include a particular feature, structure, or characteristic, but every embodiment may not necessarily include the particular feature, structure, or characteristic. Moreover, such phrases are not necessarily referring to the same embodiment or embodiments. In addition, when a particular feature, structure, or characteristic is described in connection with an embodiment or embodiments, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments. Thus, it will be appreciated that embodiments of the present disclosure may employ any combination of features described herein. All such combinations or sub-combinations of features are within the scope of the present disclosure.

The principles, representative embodiments, and modes of operation of the present disclosure have been described in the foregoing description. However, aspects of the present disclosure, which are intended to be protected, are not to be construed as limited to the particular embodiments disclosed. The embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure, as claimed.

1. A care set comprising a peripheral venous catheter, wherein the care set is needle-free.

2. The care set according to claim 1, consisting of a sterile sealed package enclosing one or more care tools including the catheter.

3. The care set according to claim 2, wherein the care tool or tools comprise a syringe.

4. The care set according to claim 3, wherein the catheter is fitted onto a dispensing end of the syringe.

5. The care set according to claim 4, wherein the catheter comprises a tube extending from a reflux chamber and wherein the dispensing end of the syringe enters the reflux chamber of the catheter.

6. The care set according to claim 4, wherein the catheter is directly attached to the dispensing end of the syringe.

7. The care set according to claim 6, wherein the catheter and the syringe are cast in one piece.

8. The care set according to claim 2, wherein the care tool or tools comprise at least one of scissors, forceps, compresses, a sterile field, a wick and a stylet.

9. The care set according to claim 2, wherein:
the package is compartmentalised and comprises a first compartment which is oversized in relation to the dimensions of the catheter, and
the catheter is arranged in this compartment.

10. The care set according to claim 9, wherein at least one of the care tools is arranged in the first compartment with the catheter.

11. The care set consisting of a sterile closed package enclosing one or more care tools including a peripheral venous catheter, the other possible care tool or tools being chosen from a syringe, scissors, forceps, compresses, a sterile field, a wick, and a stylet.

12. The care set according to claim 11, wherein the care tool or tools comprise a syringe, the catheter being fitted in and/or attached to a dispensing end of the syringe.

13. The care set according to claim 12, wherein the catheter comprises a tube extending from a reflux chamber and wherein the dispensing end of the syringe enters the reflux chamber of the catheter.

14. The care set according to claim 12, wherein the catheter and the syringe are cast in one piece.

15. A method of preparing a care set prior to wound care, wherein the care set consists of a sterile sealed package enclosing one or more care tools including:
a peripheral venous catheter comprising a tube extending from a reflux chamber, and
a syringe comprising a dispensing end,
wherein the care set is needle-free,
wherein the syringe and the catheter are two separate care tools, and wherein the method comprises the following steps:
(i) opening the package;
(ii) taking the catheter and the syringe;
(iii) directly fitting and/or attaching the dispensing end into the reflux chamber;
(iv) withdrawing a quantity of care fluid into the syringe from a free end of the catheter tube.

16. A wound care method comprising:
obtaining a care set consisting of a sterile sealed package enclosing one or more care tools including:
a peripheral venous catheter comprising a tube extending from a reflux chamber, and
a syringe comprising a dispensing end,
wherein the care set is needle-free,
wherein the syringe and the catheter are two separate care tools;
preparing the care set according to the method of claim 15;
cleaning and/or curing a wound by dispensing the quantity of care fluid from the tube of the catheter.

17. A method of preparing a care set prior to wound care, wherein the care set consists of a sterile sealed package enclosing one or more care tools including:
a peripheral venous catheter comprising a tube extending from a reflux chamber, and
a syringe comprising a dispensing end,
wherein the care set is needle-free,
wherein the catheter and the syringe are cast in one piece so that the reflux chamber continuously extends the dispensing end,
and wherein the method comprises the following steps:
(i) opening the package;
(ii) taking the catheter and the syringe;
(iv) withdrawing a quantity of care fluid into the syringe from a free end of the catheter tube.

18. A wound care method comprising:
obtaining a care set consisting of a sterile sealed package enclosing one or more care tools including:
a peripheral venous catheter comprising a tube extending from a reflux chamber, and
a syringe comprising a dispensing end,
wherein the care set is needle-free,
wherein the catheter and the syringe are cast in one piece so that the reflux chamber continuously extends the dispensing end;
preparing the care set according to the method of claim 17;
cleaning and/or curing a wound by dispensing the quantity of care fluid from the tube of the catheter.
